(19) United States
(12) Patent Application Publication
(10) Pub. No.: US 2026/0250217 A1
(43) Pub. Date: Aug. 27, 2026

(54) REACTOR FOR SYNTHESIS OF METHANE AND / OR METHANOL FROM REACTANTS AND METHOD FOR SYNTHESIS OF METHANE FROM REACTANTS

(71) Applicant: TURN2X GMBH, München (DE)

(72) Inventor: Dominik Schollenberger, Frankenthal (DE)

(21) Appl. No.: 19/162,554

(22) PCT Filed: Mar. 1, 2024
(86) PCT No.: PCT/EP2024/055376

§ 371 Date: Sep. 5, 2025

(30) Foreign Application Priority Data
Mar. 7, 2023 (CH) CH000241/2023

(51) Int. Cl. C07C 1/04; B01J 19/00; C07C 1/12; C07C 9/04; C07C 29/152; C07C 31/04

(52) U.S. Cl. CPC C07C 1/041; B01J 19/0013; C07C 1/048; C07C 1/12; C07C 9/04; C07C 29/152; C07C 31/04

(57) ABSTRACT

The present disclosure relates to a reactor (1) and to a method using such a reactor (1) for synthesis of methane and/or methanol from reactants, the reactor (1) comprises a channel shaped reactor wall (6), which provides a channel (10) for a stream of the reactants, at least one catalyst (2) arranged within the channel shaped reactor wall (6) and comprising a catalyst body structure (3), which is configured to enable the reactants to stream through the catalyst (2), wherein the at least one catalyst (2) determines at least partially a reaction zone for the syn-thesis, and a contact body (7), which is arranged between the channel shaped reactor wall (6) and the at least one catalyst (2), and which is configured to contact the channel shaped reactor wall (6) and the at least one catalyst (2) for transferring heat from the at least one catalyst (2) to the reactor wall (6) during operation of the reactor (1).

### FIELD OF THE DISCLOSURE

The present disclosure relates to a reactor for the synthesis of methane and/or methanol from reactants, to a reactor assembly and to a method for synthesis of methane and/or methanol from reactants. In particular, the present disclosure relates to a reactor for synthesis of methane from reactants comprising a channel shaped reactor wall, at least one catalyst and a contact body and to a method for synthesis of methane and/or methanol from reactants using such a reactor.

### BACKGROUND OF THE DISCLOSURE

The synthesis of methane (methanation) from CO2 (as well as CO) is a strongly exothermic reaction that requires efficient heat removal from the reaction zone, particularly when carried out catalytically with short reaction times. Conventionally, a fixed-bed reactor is used for the synthesis of methane. The fixed-bed reactor comprises a catalyst pellet bed, in which the synthesis of methane from CO2 and/or CO is performed. The pellet bed of the fixed-bed reactor defines the reactor zone. The strong exothermic synthesis of methane requires to control the heat removal from the reaction zone for controlling the temperature inside the reactor zone. Controlling of the temperature inside the reactor zone is important for ensuring a high quality product and for avoiding any damages of the reactor, in particular of the catalyst, due to surpassing of heat thresholds within the reactor. The same applies to the synthesis of methanol.

An alternative to the fixed-bed reactors are reactors with a plurality of channels, which comprise a catalyst coating. These reactors comprise a solid body structure, which define the channels. The reactants stream through the channels and react in the channels with the catalyst coating. The reaction zone is therefore within the channels. Heat removal of the reaction zone of these reactors is even more critical.

The document DE 10 2016 125 641 A1 discloses for example a process for pro-duction of a natural gas substitute from hydrogen-containing gas mixtures using a reactor with channels.

The removal of heat is conventionally performed via different coolant applications, which surround the reaction zone for heat removal. The reactor is for example surrounded by pipes in which coolant streams along during operation of the reactor. The heat removal of such an application is due to deviations from tolerances of pipes, which are for example arranged within each other not sufficient, which could result in material failure of the solid body structure of the reactor. One known option to reduce the produced heat during the synthesis is to limit the number of channels and/or cross section area of the channels such that the produced heat of the reaction does not surpass a predefined temperature value. This option is not preferred because it also limits the efficiency of the reactor. Overall, the currently used cooling applications are not sophisticated for a desired fast and reliable synthesis of methane and/or methanol from reactants.

### SUMMARY OF THE DISCLOSURE

It is an object of the present disclosure to provide a reactor and a reactor assembly for synthesis of methane and/or methanol from reactants and a method for synthesis of methane and/or methanol from reactants. In particular, it is an object of the present disclosure to provide a reactor for synthesis of methane and/or methanol from reactants and a method for synthesis of methane and/or methanol from reactants not having at least some of the disadvantages of the prior art. Advantageously, it is an object to provide a reactor allowing for efficient heat removal.

According to the present disclosure, these objects are addressed by the features of the independent claims. In addition, advantageous embodiments follow from the dependent claims and the description.

According to the present disclosure, a reactor for a synthesis from reactants, in particular for the synthesis of methane and/or methanol, is specified. The reactor comprises a channel shaped reactor wall, which provides a channel for a stream of the reactants during operation of the reactor. The reactor further comprises at least one catalyst arranged within the channel shaped reactor wall, wherein the catalyst comprises a catalyst body structure, which is configured to enable the reactants to stream through the catalyst, wherein the at least one catalyst determines at least partially a reaction zone for the synthesis. The reactor further comprises a contact body, which is arranged between the channel shaped reactor wall and the at least one catalyst, and which is configured to contact the channel shaped reactor wall and the at least one catalyst for transferring heat from the at least one catalyst to the reactor wall during operation of the reactor.

The channel shaped reactor wall has for example a circular or angular cross section, thereby forming for example a circular or angular channel. The channel extends along a longitudinal axis of the reactor, thereby forming a circular or angular cylindrical shape of the channel. Other shapes are also conceivable. The reactor wall provides thereby the channel for the stream of the reactants streaming through the channel when the reactor is in operation. The reactants typically include carbon dioxide and/or carbon monoxide. The reactants typically further include hydrogen as reducing agent. The product of the synthesis is for example methane and water (gaseous). The product of the synthesis may also be methanol and water (gaseous).

In an embodiment, the reactor is configured for the synthesis from reactants, in particular for the synthesis of methane and/or methanol. In an embodiment, the reactor is configured for the synthesis of hydrocarbons, wherein the hydrocarbons may optionally be substituted with one or more OH groups. As an example, the reactor may be configured for the synthesis of C(1-6) hydrocarbons, wherein the hydrocarbons may optionally be substituted with one or more OH groups. As an example, the reactor may be configured for the synthesis of CₙH₂ₙ₊₁R, wherein R is H or OH. As an example, the reactor may be configured for the synthesis of methane, ethane, propane, butane, pentane, hexane, methanol, ethanol, propanol, butanol, pentanol and/or hexanol, preferably methane and/or methanol, even more preferably methane.

Typically, the methanation is carried out at temperatures in a range from 150° C. to 300° C., preferably in the range from 200° C. to 250° C. Maximum temperatures within the reactor may reach 550° C. to 600° C.

The catalyst comprising the catalyst body structure is arranged within the reactor wall, such that the reactants are forced to flow through the catalyst during operation of the reactor. In an embodiment, the body structure itself is the catalyst. In other words, the body structure itself is catalytically active. In another embodiment, the body structure is coated with a catalytically active layer. In this embodiment, the body structure provides the desired surface area for the reaction, but the coating provides the catalytic functionality. The catalytically active material, for example the coating or the body structure itself, comprises for instance a washcoat, which comprises for instance porous aluminum oxide for a surface increase, and the catalytically active substances. The catalytically active substances are for example based on nickel, platinum, rhodium and/or palladium or a combination thereof. In case of coating, the coated body structure, the carrier, may be formed out of or comprises aluminum oxide (AL2O3).

The body structure is for example made of a temperature resistant ceramics like cordierite or preferably metals.

The catalyst may comprise a surrounding catalyst body wall, for example a sheet metal wall, having preferably a cylindrical shape extending along the longitudinal axis thereby surrounding at least partially or entirely the catalyst body structure in circumferential direction. The catalyst body wall thereby may form part of the catalyst body structure. The longitudinal ends of the catalyst are preferably entirely uncovered for an advantageous inflow and outflow of the reactants. The catalyst body wall comprises for example the catalytically active material or is coated with a catalytically active laver.

The contact body arranged between the channel shaped reactor wall and the at least one catalyst contacts the channel shaped reactor wall and the at least one catalyst, for example the surrounding catalyst body wall or the catalyst body structure, for transferring heat from the at least one catalyst to the reactor wall. It is understood that the contact body typically contacts the at least one catalyst contacts the channel shaped reactor directly, although it may also contact them indirectly, e.g. through the intermediacy of one or more heat-transferring layers arranged between the contact body and the channel shaped reactor wall and/or between the contact body and the catalyst body structure. The contact body provides an advantageous heat transfer from the catalyst to the reactor wall due to a controlled heat conduction from the catalyst to the contact body and from the contact body to the reactor wall. The contact body is preferably made of a material of relatively high thermal conductivity like metals (steel, aluminium etc.). The heat transferred by the contact body to the reactor wall may further be transferred away by additional applications arranged radially outside of the reactor wall, such as coolants. Further, the contact body enables to position the catalyst advantageously within the channel shaped reactor wall compared to conventional reactors in which the catalyst is for example inserted in the reactor wall such that the catalyst contacts randomly the reactor wall. The contact body is a thermal heat bridge between the catalyst and the reactor wall.

Overall, the contact body advantageously increases the heat transfer from the catalyst to the reactor wall, makes the heat transfer controllable and enables an advantageous positioning of the catalyst within the reactor wall independently from manufacturing tolerances of the different parts, like the catalyst diameter or the reactor wall inner diameter.

In an embodiment, the reactor comprises a plurality of catalysts, which are arranged in series with respect to each other. The reactor preferably has an elongated cylindrical shape, along which the reactants flow during operation. The shape is for example predefined by the channel shaped reactor wall, which is for example a tube or a pipe. The catalyst with a predefined axial extension does for example not entirely extend along the channel shaped reactor wall. Therefore, a plurality of catalysts may for example be arranged within the channel shaped reactor wall in axial direction next to each other, thereby arranged in series with respect to each other. The reactants flow, for instance through a first catalyst and afterwards through a second catalyst arranged downstream with respect to the first catalyst during operation of the reactor. This embodiment increases the reactor zone for an advantageous synthesis. In an embodiment, the plurality of catalysts contact each other on one axial end. In another embodiment, the plurality of catalysts are separated in axial direction with respect to each other by an intermediate zone, which enables mixture of the reactants after streaming out of a downstream arranged catalyst prior of streaming into an upstream arranged catalyst. In an embodiment, the contact body is configured to position the plurality of catalysts with respect to its axial position within the reactor. In other words, the different catalysts are arranged on the contact body such that the predefined position of the different catalysts (with or without intermediate zone) is realized as desired within the reactor.

In an embodiment, the reactor wall and the at least one catalyst are arranged coaxially with respect to each other, which is realized by the contact body. In this embodiment, the contact body is configured to position the catalyst or the plurality of catalysts coaxially with respect to the reactor wall, which improves the stream of reactants through the reactor. In a further embodiment, the plurality of catalysts are arranged coaxially with respect to each other by the contact body. The contact body extends for example along the plurality of catalysts such that the plurality of catalysts are coaxially aligned with each other, which improves the stream of reactants from one catalyst to the next catalyst during operation of the reactor.

In an embodiment, the contact body comprises or is a spring element, which is configured to exert a spring force on the at least one catalyst and/or the reactor wall when arranged between the channel shaped reactor wall and the at least one catalyst. The spring element is for example provided via a specific protrusion on the contact body. The spring element is for example a sheet metal arranged on the contact body configured to be elastically deformed when arranged between the reactor wall and the at least one catalyst. In another embodiment, the entire contact body is for example made of a sheet metal, which is shaped such it forms one or a plurality of the spring elements. The spring force acting on the reactor wall and the at least one catalyst advantageously positions the at least one catalyst or the plurality of catalysts rigidly within the reactor wall during operation of the reactor. Typically, the contact body has a higher radial compressibility than the catalyst, in particular than the catalyst body structure. The contact body is typically made of a material able to withstand temperatures up to 800° C., preferably up to 700° C.

In an embodiment, the contact body is made at least partially of a catalytically active material or comprises a contact body coating made at least partially of a catalytically active coating, which is configured to function as bypass catalyst for a bypass flow of the reactants, which flows along the contact body thereby by-passing the at least one catalyst during operation of the reactor and contacting the bypass catalyst, which is the coating of the contact body. A gas gap is present between the reactor wall and the at least one catalyst in which the contact body is positioned. The gas gap has for example an extension from 1 mm to 3 mm. The gas gap enables the flow of reactants to bypass partially the catalyst. In this embodiment, the reactor zone is extended also into the gas gap because the contact body itself or its coating provide the catalytically active material for the synthesis.

In an embodiment, the catalyst body structure forms a plurality of catalyst channels, which are configured to enable the reactants to flow through the catalyst during operation of the reactor. The number of channels is for example in a range from 100 to 1000, preferably in a range from 250 to 450. The channels advantageously increase the available surface within the catalyst for an advantageous synthesis. The channels have for example a circular, an angular, a rectangular, a squared and/or a honeycomb shape.

In an embodiment, the contact body is arranged via an adhesive joint on the reactor wall and/or the at least one catalyst. The adhesive joint is for example manufactured via gluing, soldering or welding. In other words, after positioning the reactor wall, the at least one catalyst and the contact body, the contact body is fixedly attached to the other parts via the adhesive joint. The adhesive joint is for example a (soldering, welding) seam extending in axial direction and/or in circumferential direction, preferably in a helical manner, along the contact body and the reactor wall and/or the catalyst.

In an embodiment, the contact body comprises a plurality of contacting element, which are distributed, preferably evenly, around the at least one catalyst, wherein the contacting elements form the contact body. In this embodiment, the contact body is formed out of the plurality of the contacting elements. The contacting elements are for example elongated sheet metal parts, which have a ring cylindrical or half ring cylindrical cross section. The plurality of the contacting elements is for example inserted between the reactor wall and the at least one catalyst, such that an elastic deformation of the contacting elements is caused. The elastic deformation exerts the spring force on the reactor wall/the at least one catalyst for an advantageous positioning of the catalyst. The contacting elements are for example fixedly arranged via the adhesive joint.

In an embodiment, the contact body extends in circumferential direction around the at least one catalyst and comprises first protrusions, which contact the catalyst, and second protrusions, which contact the reactor wall. The protrusions may form at least partially the spring elements of the contact body. In an embodiment, the contact body is compressed between the at least one catalyst and the reactor wall in an assembled state. In other words, the contact body, the catalyst and the reactor wall are dimensioned such that, when the contact body is arranged between the at least one catalyst and the reactor wall, an inner contact contour and an outer contact contour of the contact body are each loaded with a radial compression force. The inner contact contour is defined by the first protrusions and the outer contact contour is defined by the second protrusions.

In an embodiment, wherein the contact body has a wave like shape or star like shape, in particular the cross section of the contact body has the wave like shape or the star like shape. In case of a wave like shape, the wave crests are preferably configured to contact the reactor wall and the wave troughs are preferably configured to contact the at least one catalyst or vice versa. The wave like shape is for example realized by the contact body being made of a corrugated sheet metal. In case of a star like shape, the star tips are preferably configured to contact the reactor wall and the star foundations are preferably configured to contact the at least one catalyst or vice versa. The contact body may has a sinus like shape thereby forming waves. Other profiles are also conceivable.

In an embodiment, the contact body comprise an oversize, with respect to the radial dimensions of the reactor wall and the at least one catalyst, wherein the oversize forces the contact body to deform, preferably elastically, when inserted between the reactor wall and the at least one catalyst. The deformation creates a larger contact portion of the contact body between the reactor wall and the at least one catalyst compared to a contact body without oversize. Further, the oversize enables an advantageous simple realization of the spring element of the contact body.

In an embodiment, the thickness of the contact body varies along its circumferential direction, with respect to the at least one catalyst, such that the portions of the contact body, which are configured to contact the reactor wall and the at least one catalyst are thinner compared to portions of the contact body which connect the contact portions. In other words, the portions of the contact body or the portions of the contact elements, which, when inserted, contacts the reactor wall and/or the catalyst are thinner compared to the portion of the contact body or the portion of the contact elements which, when inserted, connect the contacting portions. The thin portions enable a relatively simple deformation such that the contact surface is increased, which enables an advantageous heat transfer from the catalyst to the contact body and the contact body to the reactor wall, and the relatively thick portion of the contact body enable that the heat transfer along the contact body is improved.

In an embodiment, the contact body contacts the radial outer surface of the at least one catalyst in a range from 5% to 25%, preferably 8% to 15%, even more preferably 10% to 12% of the respective surface and/or wherein the contact body contacts the radial inner surface of the reactor wall in a range from 5% to 25%, preferably 8% to 15%, even more preferably 10% to 12% of the respective surface. In other words, the contact body contact the radial outer surface of the at least one catalyst in a range from 5% to 25% with respect to the available radial surface of the catalyst. The contact body contact the radial inner surface of the reactor wall in a range from 5% to 25% with respect to the available radial inner surface of the reactor wall. The specified contact surfaces enable an advantageous heat transfer from the catalyst to the contact body.

In an embodiment, the reactor further comprising a cooling wall, which surrounds the reactor wall such that a cooling channel is formed between the reactor wall and the cooling wall, wherein the cooling channel is configured to enable a coolant to flow along the cooling channel during operation of the reactor. The coolant, for example a cooling oil or water, contacts at least partially the radial outer surface of the reactor wall, such that heat, which is guided from the reaction zone via the contact body to the reactor wall, can be transferred into the coolant via heat convection. The coolant wall enables advantageously to remove the heat from the reactor wall.

In a further aspect of the present disclosure, a reactor assembly is specified which comprises a plurality of reactors as described above and hereinafter. The reactor assembly preferably comprises a plurality of reactors arranged in parallel with respect to each other, which increases the output of the reactor assembly during its operation.

In a further aspect of the present disclosure, a method for synthesis is specified, in particular of methane and/or methanol from reactants. The method comprises the steps of:
a. Providing a reactor described above and hereinafter;
b. Providing an inflow of the reactants into the provided reactor for the synthesis of methane and/or of methanol.

In an embodiment, the synthesis of methane involves methanation of carbon dioxide and/or carbon monoxide. It is typically carried out at temperatures up to 600° C. Preferably, the reaction is controlled such that the temperature inside the catalyst does not exceed 700° C., preferably such that it does not exceed 650° C., preferably such that it does not exceed 600° C., preferably such that it does not exceed 550° C., preferably such that it does not exceed 500° C.

### BRIEF DESCRIPTION OF THE DRAWINGS

The present disclosure will be explained in more detail, by way of example, with reference to the figures in which:

FIG. 1 shows a perspective view of the reactor according to a first exemplary embodiment;

FIG. 2 shows a top view of the reactor according to the first exemplary embodiment;

FIG. 3 shows a longitudinal cross section of the reactor according to the first exemplary embodiment;

FIG. 4 shows a schematic cross section of a reactor according to a second exemplary embodiment;

FIG. 5 shows in a perspective view a contact body according to a first exemplary embodiment as arranged in the reactor according to the first exemplary embodiment;

FIG. 6 shows in a perspective view a contact body according to a second exemplary embodiment;

FIG. 7 shows in a perspective view a contact body according to a third exemplary embodiment;

FIG. 8 shows in a top view the contact body according to the third exemplary embodiment.

FIG. 9 shows in a perspective view a catalyst according to a first exemplary embodiment;

FIG. 10 shows in a perspective view row material for a contact body;

FIG. 11 shows a diagram, which compares the temperature profiles within a catalyst along its longitudinal axis of a reactor with and without a contact body.

### DETAILED DESCRIPTION OF THE DRAWINGS

Reference will now be made in detail to certain embodiments, examples of which are illustrated in the accompanying drawings, in which some, but not all features are shown. Indeed, embodiments disclosed herein may be embodied in many different forms and should not be construed as limited to the embodiments set forth herein; rather, these embodiments are provided so that this disclosure will satisfy applicable legal requirements. Whenever possible, like reference numbers will be used to refer to like components or parts.

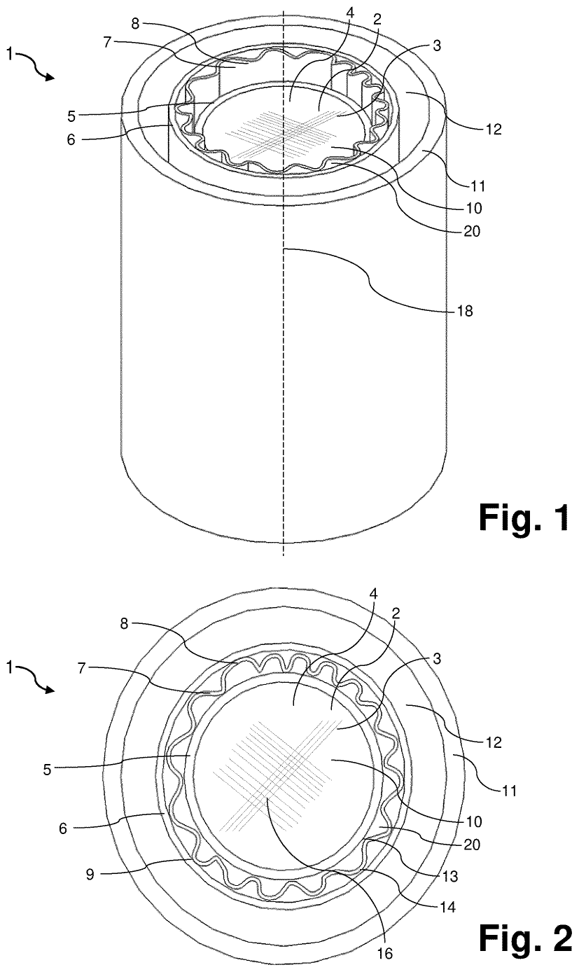

The FIGS. 1, 2 and 3 show a first exemplary embodiment of a reactor 1. The reactor 1 comprises a catalyst 2, which comprises a catalyst body structure 3 defining a plurality of channels 16 within the catalyst 2, along which the reactants stream during operation of the reactor 1. The catalyst body structure 3 defines at least partially the reaction zone of the catalyst 2 in which the synthesis of methane takes place during operation of the reactor 1. In this embodiment, the catalyst body structure 3 comprises a catalyst coating 4, which comprises the catalytically active materials. The catalyst body structure 3 provides the desired large surface and the coating 4 provides the catalytically active materials. The catalyst body structure 3 of the catalyst 2 has a circular cylindrical shape (other shapes are also conceivable) and is surrounded on its shell surface by a catalyst body wall 5, which is for example made of sheet metal. The reactor 1 as presented in the figures further comprises a channel shaped reactor wall 6, which is for example a pipe or tube and in which the catalyst 2 is positioned. The catalyst 2 and the reactor wall 6 are preferably arranged coaxially with respect to each other, as best visible in the FIGS. 1 and 2. FIG. 1 advantageously shows a longitudinal axis 18 of the reactor 1 along which the different parts of the reactor 1 extend. The reactor wall 6 provides a streaming channel 10 for the reactants for the synthesis of methane, along which the reactants stream during operation of the reactor 1.

The reactor 1 further comprises a contact body 7, which is arranged between the catalyst 2, in particular between the catalyst body wall 5, and the reactor wall 6. The contact body 7 is configured to contact both parts for transferring heat from the catalyst 2 to the reactor wall 6 during operation of the reactor 1. The contact body 7 completely surrounds the at least one catalyst 2 as best visible in FIG. 2. The contact body 7 is made of a corrugated sheet metal and contacts with first contact portions 13 the catalyst 2 and with second contact portions 14 the reactor wall 6. The contact body 7 further comprises a contact body coating 8, which comprises catalytically active materials such that a bypass flow of reactants streaming along a gas gap 20 between the reactor wall 6 and the catalyst 2 during operation of the reactor 1 also contact the contact body coating 8, thus catalyzing the chemical transformation of the reactants to methane. The contact body 7 is preferably fixedly arranged within the reactor 1 by an adhesive joint 9, for example a soldering seam or a welding seam, which extends axially or in circumferential direction (or in both directions, like a helix) between the contact body 7 and/or the catalyst 2 and the reactor wall 6. The adhesive joint 9 further advantageously increases the heat transfer between the different parts. The adhesive joint 9 further may be configured to position the catalyst 2 by the contact body 7 within the reactor 1.

The FIGS. 1, 2 and 3 further show a cooling wall 11, which surrounds the reactor wall 6 and which thereby provides a cooling channel 12 or a coolant flow during operation of the reactor 6. The cooling wall 11 is arranged preferably co-axially with respect to the catalyst 2, the contact body 7 and the reactor wall 6, which enables that the coolant channels 12 has a constant thickness in circumferential direction which improves the heat transport.

The contact body 7 as shown in the FIGS. 1, 2 and 3 has an oversize with respect to the radial outer surface of the catalyst body wall 5 and the radial inner surface of the reactor wall 6 such that at least an elastic deformation is caused when the contact body 7 is inserted into the reactor 1. The elastic deformation advantageously increases the contact surface between the contact body 7 and the catalyst 2 and the reactor wall 6, which improves the heat transfer. Further, the elastic deformation exerts a spring force from the contact body 7 on the reactor wall 6 and the catalyst 2 such that the catalyst 2 is advantageously positioned in the reactor 1 by the contact body 7.

In another embodiment, not shown in the figures, the contact body 7 may comprise protrusions, which form a spring element 15 and which are configured to deform elastically when the contact body 7 is inserted in the reactor 1, such that the spring force is exerted on the catalyst 2 for positioning the catalyst 2 within the reactor 1.

The FIGS. 1, 2 and 3 further indicate schematically catalyst channels 16, which are defined by the catalyst body structure 3 of the catalyst 2. The catalyst channels 16 have for example a circular, an angular, a rectangular, a squared or a honeycomb shape. The catalyst 2 comprises for example 250 to 500 channels determined by the catalyst body structure 3, which is for example made of ceramic or metal.

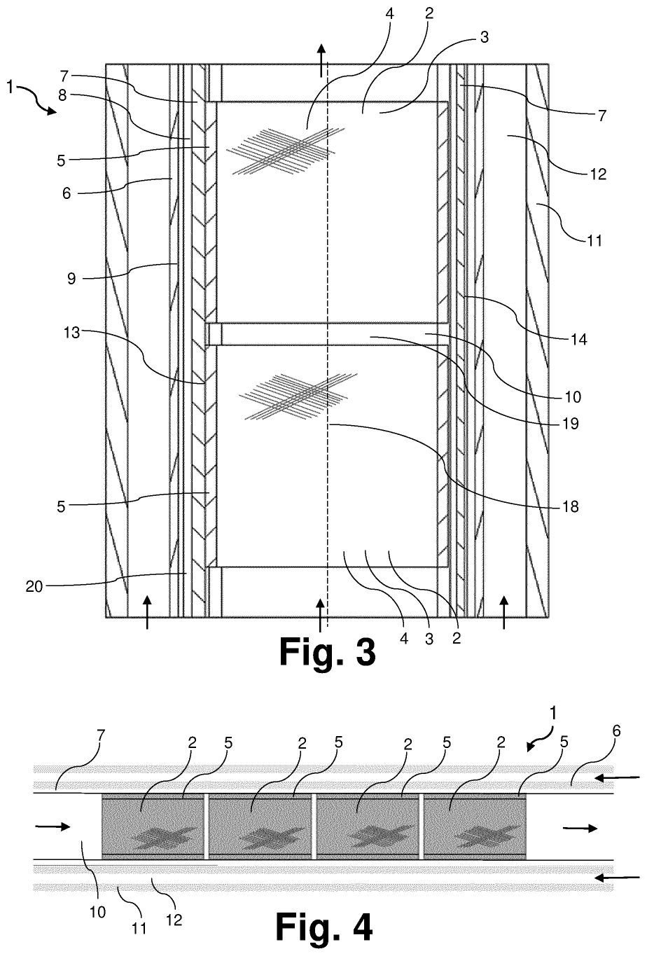

FIG. 3 advantageously shows a plurality of the catalysts 2 arranged in series with respect to each other. The catalysts 2 are arranged axially next to each other with respect to the longitudinal axis 18. The catalyst 2 arranged downstream and the catalyst 2 arranged upstream contact both the contact body 7. In other words, the contact body 7 extends along both catalysts 2 and contacts thereby both catalysts 2. Both catalysts 2 are thereby advantageously positioned coaxially with respect to each other by the contact body 7 extending along both catalysts 2.

FIG. 3 further advantageously shows the gas gap 20 between the catalysts 2 and the reactor wall 6 in which the contact body 7 is arranged. FIG. 3 further shows a streaming direction of the reactants along the reactant channel 10 and a streaming direction of the coolant within the cooling channel 12. FIG. 3 further shows that the catalysts 2 do not contact each other at its respective axial ends.

An intermediate zone 19 is arranged between the two catalysts 2 in axial direction. The intermediate zone 19 has the advantage that the reactants can mix again after outflowing from the downstream catalyst 2 and prior to inflowing into the upstream catalyst 2. In another embodiment, the catalysts 2 contact each other axially.

FIG. 4 shows the reactor 1 with a plurality of catalysts 2 within the reactor wall 6, in particular with four catalysts 2 all arranged in series with respect to each other. FIG. 4 further shows schematically the contact body 7 contacting all of the catalysts 2 thereby positioning the catalysts 2 coaxially with respect to each other and coaxially with respect to the reactor wall 6, which surrounds the catalysts 2 and the contact body 7. FIG. 4 further shows a counter-flow cooling direction of the coolant with respect to the flow direction of the reactants during operation of the reactor 1.

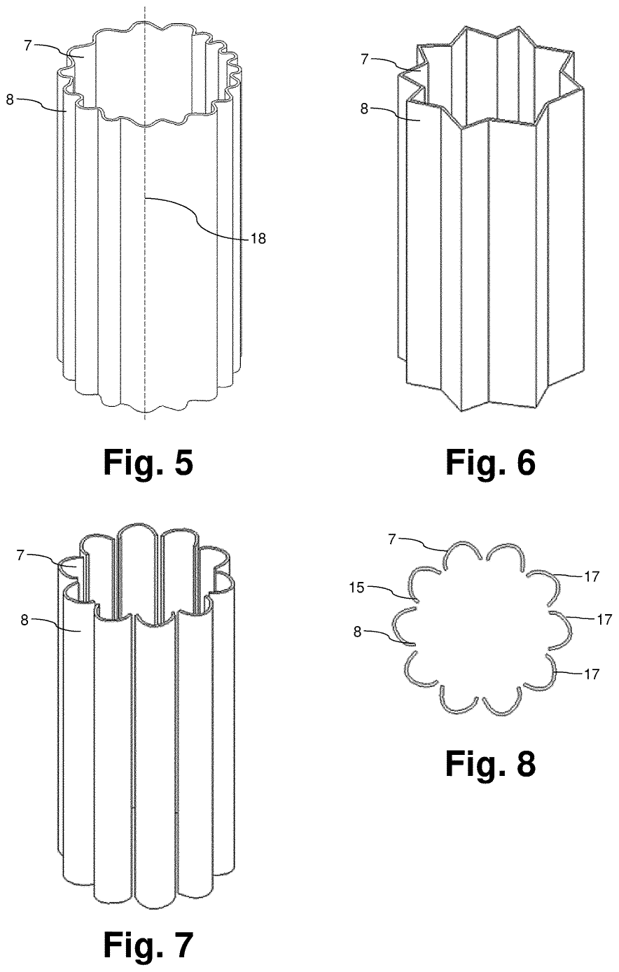

FIG. 5 shows in a perspective view the contact body 7 of the reactor 1 of FIGS. 1 to 3. The contact body 7 extends in axial direction along the longitudinal axis 18 and is for example made of a corrugated sheet metal such that the desired wave-like cross section of the contact body 7 is realized. The contact body 7 preferably comprises a contact body coating 8, which comprises the catalytically active materials.

FIG. 6 shows in a perspective view another embodiment of the contact body 7 having a star like cross section.

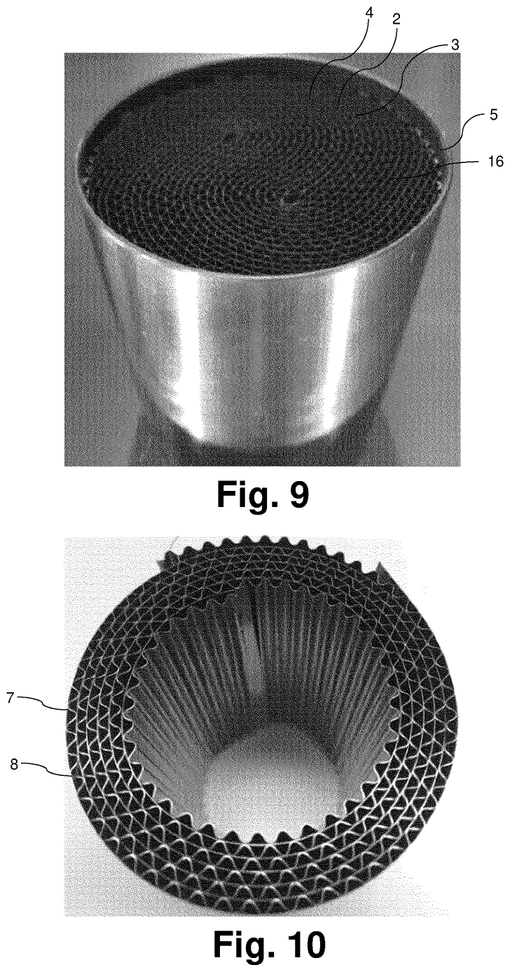

FIG. 7 and FIG. 8 show in a perspective view and in a top view another embodiment of the contact body 7, wherein the contact body 7 comprises a plurality of contacting elements 17. The contacting element 17 have a half ring cylindrical shape and are configured to be arranged between the at least one catalyst 2 and the reactor wall 6. The contacting element 17 preferably have an oversize with respect to the gas gap 20 between the at least one catalyst 2 and the reactor wall 6 such that inserting the contacting elements 17 causes an elastic deformation of the for example parts of the contacting elements 17. The elastic deformation exerts a spring force on the at least one catalyst 2 for positioning the at least one catalyst 2 within the reactor. The contacting element 17 are preferably distributed evenly around the at least one catalyst 2.

FIG. 9 show in a perspective detailed view the catalyst 2. FIG. 9 advantageously shows the catalyst body structure 3 made of thin sheet metal. The catalyst body structure 3 forms a plurality of the catalyst channels 16, along which the reactants stream during operation of the reactor 1. The catalyst body structure 3 comprise a catalyst coating 4, which comprises the catalytically active materials. FIG. 9 advantageously shows the catalyst body wall 5 enclosing the catalyst body structure 2 in circumferential direction.

FIG. 10 shows a corrugated sheet metal, which could be used to form the contact body 7.

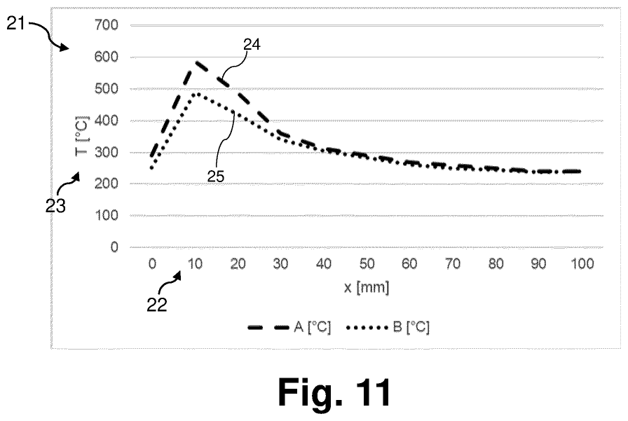

FIG. 11 shows a diagram 21 comprising two temperature profiles 24, 25 resulting from a simulation of a synthesis of methane using different reactors 1. In a first simulation, the reactor 1 does not comprise a contact body 7 arranged between the catalyst 2 and the reactor wall 6. In a second simulation, the reactor 1 does comprise a contact body 7 arranged between the catalyst 2 and the reactor wall 6. The catalyst 2 used in both simulations is the same and has an outer diameter of 80 mm and an axial length of 100 mm. The diameter of the reactor wall 6 is 82 mm resulting in a gas gap 20 of 2 mm. In the second simulation the gas gap 20 is filled with a corrugated sheet metal having a thickness of 110 micrometer as contact body 7. The corrugated sheet metal contacts 12.83 % of the radial outer surface of the catalyst 2, in particular of the catalyst body wall 5. The corrugates sheet metal contact also contact 12,83 % of the radial inner surface of the respective reactor wall 6. The heat resulting from the exothermal synthesis within the catalyst 2 is transferred in the first simulation via the gas gap 20 without contact body 7 to the reactor wall and in the second simulation with the contact body 7.

The diagram 21 shows on its abscissa 22 the axial extension of one catalyst 2 with respect to the longitudinal axis 18 and on its ordinate 23 the temperature within the catalyst 2 during the simulated operation of the reactor. The first resulting temperature profile A 24 resulting from the simulation without the contact body 7 within the reactor 1 is presented in the diagram 21 with a dashed line. The second resulting temperature profile B resulting from the simulation with the contact body 7 within the reactor 1 is presented in the diagram 21 with a dotted line.

The diagram 21 advantageously shows that the temperature within the catalyst 2 of the first simulation peaks at around 580° C. and that the temperature within the catalyst 2 of the second simulation peaks at around 480° C. The difference is roughly 100° C., which results only from the presence of the contact body 7 as thermal heat bridge. The lower temperature within the catalyst 2 is advantageous in view of the synthesis itself and in view of the durability of the catalyst.

Rather, the words used in the specification are words of description rather than limitation, and it is understood that various changes may be made without departing from the Spirit and scope of the disclosure.

### LIST OF REFERENCE SINGS

1 Reactor
2 Catalyst
3 Catalyst body structure
4 Catalyst coating
5 Catalyst body wall
6 Reactor wall
7 Contact body
8 Contact body coating
9 Adhesive joint
10 Reactant channel
11 Cooling wall
12 Cooling channel
13 First contact portion
14 Second contact portion
15 Spring element
16 Catalyst channels
17 Contacting elements
18 Longitudinal axis
19 Intermediate zone
20 Gas gap
21 Diagram
22 Abscissa
23 Ordinate
24 First temperature profile A
25 Second temperature profile B

1. Reactor (1) for synthesis of methane and/or methanol from reactants, the reactor (1) comprising:
a. a channel shaped reactor wall (6), which provides a channel (10) for a stream of the reactants;
b. at least one catalyst (2) arranged within the channel shaped reactor wall (6) and comprising a catalyst body structure (3), which is configured to enable the reactants to stream through the catalyst (2), wherein the at least one catalyst (2) determines at least partially a reaction zone for the synthesis; and
c. a contact body (7), which is arranged between the channel shaped reactor wall (6) and the at least one catalyst (2), and which is configured to contact the channel shaped reactor wall (6) and the at least one catalyst (2) for transferring heat from the at least one catalyst (2) to the reactor wall (6) during operation of the reactor (1).

2. The reactor (1) according to claim 1, wherein the reactor (1) comprises a plurality of catalysts (2), which are arranged in series with respect to each other.

3. The reactor (1) according to claim 1, wherein the contact body (7) comprises or is a spring element (15), which is configured to exert a spring force on at least one of: the at least one catalyst (2) or the reactor wall (6), when arranged between the channel shaped reactor wall (6) and the at least one catalyst (2).

4. The reactor (1) according to claim 1, wherein the contact body (7) comprises a contact body coating (8), which is configured to function as bypass catalyst for a bypass flow of the reactants, which flows along the contact body (7) thereby bypassing the at least one catalyst (2) during operation of the reactor (1) and contacting the bypass catalyst.

5. The reactor (1) according to claim 1, wherein the catalyst body structure (3) forms a plurality of catalyst channels (16), which are configured to enable the reactants to flow through the catalyst (2) during operation of the reactor (1).

6. The reactor (1) according to claim 1, wherein the contact body is arranged via an adhesive joint (9) on at least one of: the reactor wall (6) or the at least one catalyst (2).

7. The reactor (1) according to claim 1, wherein the contact body (7) comprises a plurality of contacting element (17), which are distributed around the at least one catalyst (2), wherein the contacting elements (17) form the contact body (7).

8. The reactor (1) according to claim 1, wherein the contact body (7) extends in circumferential direction around the at least one catalyst (2) and comprises first protrusions, which contact the catalyst (2), and second protrusions, which contact the reactor wall (6).

9. The reactor (1) according to claim 8, wherein the contact body (7) has a wave like form or star like form.

10. The reactor (1) according to claim 1, wherein the contact body (7) comprises an oversize, with respect to the radial dimensions of the reactor wall (6) and the at least one catalyst (2), wherein the oversize forces the contact body (7) to deform when inserted between the reactor wall (6) and the at least one catalyst (2).

11. The reactor (1) according to claim 1, wherein the thickness of the contact body (7) varies along its circumferential direction such that the portions which are configured to contact the reactor wall (6) and the at least one catalyst (2) are thinner compared to portions of the contact body (7) which connect the contact portions.

12. The reactor (1) according to claim 1, wherein the contact body (7) contacts the radial outer surface of the at least one catalyst (2) in a range from 5% to 25%, and/or wherein the contact body (7) contacts the radial inner surface of the reactor wall (6) in a range from 5% to 25%.

13. The reactor (1) according to claim 1, the reactor (1) further comprising a cooling wall (11), which surrounds the reactor wall (6) such that a cooling channel (12) is formed between the reactor wall (6) and the cooling wall (11), wherein the cooling channel (12) is configured to enable a coolant to flow along the cooling channel (12).

14. Reactor assembly comprising a plurality of reactors (1) as claimed in claim 1, wherein the plurality of reactors (1) is arranged in parallel with respect to each other.

15. Method for synthesis of methane and/or methanol from reactants, the method comprising the steps of:
a. Providing (S1) a reactor (1) as claimed in claim 1;
b. Providing (S2) an inflow of the reactants into the provided reactor for the synthesis of methane and/or methanol.
